(19) United States
(12) Patent Application Publication
(10) Pub. No.: US 2024/0054645 A1
(43) Pub. Date: Feb. 15, 2024

(54) MEDICAL IMAGE PROCESSING APPARATUS, MEDICAL IMAGE PROCESSING METHOD, AND PROGRAM

(71) Applicant: FUJIFILM Corporation, Tokyo (JP)

(72) Inventor: Katsuyuki HIGA, Kanagawa (JP)

(73) Assignee: FUJIFILM Corporation, Tokyo (JP)

(21) Appl. No.: 18/496,907

(22) Filed: Oct. 29, 2023

Related U.S. Application Data
(63) Continuation of application No. PCT/JP2022/014344, filed on Mar. 25, 2022.

(30) Foreign Application Priority Data
May 13, 2021 (JP) 2021-081548

(51) Int. Cl. G06T 7/00; G06T 5/50; G06V 10/25; G06V 10/764; G16H 30/40

(52) U.S. Cl. CPC G06T 7/0012; G06T 5/50; G06V 10/25; G06V 10/764; G16H 30/40; G06T 2207/10132; G06T 2207/20221

(57) ABSTRACT

A processor of a medical image processing apparatus performs: an image acquisition process of acquiring a medical image; a region information acquisition process of acquiring region information regarding a plurality of regions of interest included in the medical image, the region information including positions and category classifications of the plurality of regions of interest; a display control process of superimposing a plurality of pieces of graphic information that indicate the results of the category classifications of the plurality of regions of interest on the medical image and causing a display unit to display the plurality of pieces of graphic information; and a superimposed-position determination process of determining superimposed positions of the plurality of pieces of graphic information to be displayed by the display control process, on the basis of a relative positional relationship between the plurality of regions of interest.

### CROSS-REFERENCE TO RELATED APPLICATIONS

The present application is a Continuation of PCT International Application No. PCT/JP2022/014344 filed on Mar. 25, 2022 claiming priority under 35 U.S.C § 119(a) to Japanese Patent Application No. 2021-081548 filed on May 13, 2021. Each of the above applications is hereby expressly incorporated by reference, in its entirety, into the present application.

### BACKGROUND OF THE INVENTION

### 1. Field of the Invention

The present invention relates to a medical image processing apparatus, a medical image processing method, and a program and specifically relates to a medical image processing apparatus, a medical image processing method, and a program for superimposing on a medical image and displaying information regarding a region of interest in the medical image.

### 2. Description of the Related Art

In recent years, the practice of automatically detecting a region of interest, such as an organ or a lesion, present in a medical image by using AI (artificial intelligence) and displaying information (graphic information) regarding the detected region of interest on a monitor to assist a doctor in medical examination and diagnosis has been used.

For example, JP2011-206168A describes a technique for superimposing on an endoscopic image and displaying findings made by a doctor or the like to assist the doctor in medical examination and diagnosis using an endoscope.

### SUMMARY OF THE INVENTION

A plurality of regions of interest may be detected in a medical image. When a plurality of regions of interest are detected, pieces of graphic information to be displayed may be displayed while overlapping each other depending on the positions of the detected regions of interest. Further, depending on the positions of the detected regions of interest, graphic information may be displayed while overlapping a region of interest, and the region of interest, which is an observation target, may be hidden. When the graphic information is thus displayed, the region of interest and the graphic information are difficult to be viewed.

The present invention has been made in view of the above-described circumstances, and an object thereof is to provide a medical image processing apparatus, a medical image processing method, and a program capable of displaying a plurality of regions of interest and pieces of graphic information on a medical image so as to be easily viewable.

To achieve the above-described object, a medical image processing apparatus according to an aspect of the present invention is a medical image processing apparatus including: a processor, the processor being configured to perform: an image acquisition process of acquiring a medical image; a region information acquisition process of acquiring region information regarding a plurality of regions of interest included in the medical image, the region information including positions and category classifications of the plurality of regions of interest; a display control process of superimposing a plurality of pieces of graphic information that indicate the category classifications of the plurality of regions of interest on the medical image and causing a display unit to display the plurality of pieces of graphic information; and a superimposed-position determination process of determining superimposed positions of the plurality of pieces of graphic information to be displayed by the display control process, on the basis of a relative positional relationship between the plurality of regions of interest.

According to this aspect, the superimposed position of each piece of graphic information to be displayed by the display control process is determined on the basis of the relative positional relationship between the plurality of regions of interest to thereby allow easily viewable display of the regions of interest and the pieces of graphic information.

Preferably, the processor is configured to perform a region information generation process of generating the region information by detecting the plurality of regions of interest included in the medical image and estimating the category classifications of the plurality of detected regions of interest.

Preferably, at least one of the plurality of regions of interest is an anatomical region.

Preferably, at least one of the plurality of regions of interest is an annotation drawn by a user on the medical image.

Preferably, in the superimposed-position determination process, a superimposed position of a piece of graphic information of at least one region of interest among the plurality of regions of interest is determined on the basis of a position of another region of interest among the plurality of regions of interest.

Preferably, in the superimposed-position determination process, a superimposed position of a piece of graphic information of at least one region of interest among the plurality of regions of interest is determined on the basis of a superimposed position of a piece of graphic information of another region of interest among the plurality of regions of interest.

Preferably, in the image acquisition process, a plurality of successive time-series medical images are acquired.

Preferably, in the superimposed-position determination process, among the plurality of medical images, a superimposed position of graphic information in a current medical image is determined on the basis of a superimposed position of the graphic information in a past medical image.

Preferably, in the superimposed-position determination process, as the superimposed position of the graphic information in the current medical image, a position in a region that is within a distance of a first threshold value from the superimposed position of the graphic information in the past medical image is chosen.

Preferably, in the display control process, a leader line that extends up to a superimposed position of a piece of graphic information of at least one region of interest among the plurality of regions of interest is displayed.

Preferably, the processor is configured to perform an inclusion relationship acquisition process of acquiring inclusion relationship information about the plurality of regions of interest on the basis of the region information, and in the superimposed-position determination process, the superimposed positions of the plurality of pieces of graphic information are determined on the basis of the inclusion relationship information.

Preferably, in the superimposed-position determination process, whether to display a leader line for at least one region of interest among the plurality of regions of interest is determined on the basis of the inclusion relationship information to switch between display and non-display.

Preferably, in the display control process, pieces of graphic information of regions of interest having an inclusion relationship are displayed in a nested form that indicates the inclusion relationship, on the basis of the inclusion relationship information.

Preferably, in the display control process, pieces of information that indicate bounds of the regions of interest are displayed on the basis of the region information.

Preferably, the pieces of information indicating the bounds of the regions of interest are bounding boxes, and in the display control process, the pieces of graphic information are displayed so as to correspond to the bounding boxes.

Preferably, each of the pieces of graphic information is constituted by text information that indicates a corresponding one of the category classifications.

A medical image processing method according to another aspect of the present invention is a medical image processing method using a medical image processing apparatus including a processor, the medical image processing method including: an image acquisition step of acquiring a medical image; a region information acquisition step of acquiring region information regarding a plurality of regions of interest included in the medical image, the region information including positions and category classifications of the plurality of regions of interest; a display control step of superimposing a plurality of pieces of graphic information that indicate the category classifications of the plurality of regions of interest on the medical image and causing a display unit to display the plurality of pieces of graphic information; and a superimposed-position determination step of determining superimposed positions of the plurality of pieces of graphic information to be displayed in the display control step, on the basis of a relative positional relationship between the plurality of regions of interest, the steps being performed by the processor.

A program according to another aspect of the present invention is a program for causing a medical image processing apparatus including a processor to perform a medical image processing method, the program causing the processor to perform: an image acquisition step of acquiring a medical image; a region information acquisition step of acquiring region information regarding a plurality of regions of interest included in the medical image, the region information including positions and category classifications of the plurality of regions of interest; a display control step of superimposing a plurality of pieces of graphic information that indicate the category classifications of the plurality of regions of interest on the medical image and causing a display unit to display the plurality of pieces of graphic information; and a superimposed-position determination step of determining superimposed positions of the plurality of pieces of graphic information to be displayed in the display control step, on the basis of a relative positional relationship between the plurality of regions of interest.

According to this aspect, the superimposed position of each piece of graphic information to be displayed by the display control process is determined on the basis of the relative positional relationship between the plurality of regions of interest to thereby allow easily viewable display of the regions of interest and the pieces of graphic information.

### BRIEF DESCRIPTION OF THE DRAWINGS

FIG. 1 is a schematic diagram illustrating an overall configuration of an ultrasonic endoscope system equipped with a medical image processing apparatus;

FIG. 2 is a block diagram illustrating an embodiment of an ultrasound processor device;

FIG. 3 is a diagram illustrating an example of display of regions of interest and bounding boxes;

FIG. 4 is a diagram for explaining an example of display of graphic information in the related art;

FIG. 5 is a diagram for explaining another example of display of graphic information in the related art;

FIG. 6 is a diagram illustrating an example of display of graphic information;

FIG. 7 is a flowchart illustrating a medical image processing method;

FIG. 8 is a block diagram illustrating an embodiment of the ultrasound processor device;

FIG. 9 is a diagram illustrating an example display form;

FIG. 10 is a diagram illustrating another example display form;

FIG. 11 is a diagram for explaining display of graphic information on an ultrasound image P1 and an ultrasound image P2;

FIG. 12 is a diagram for explaining display of graphic information on the ultrasound image P1 and the ultrasound image P2; and

FIG. 13 is a diagram for explaining an example display form.

### DESCRIPTION OF THE PREFERRED EMBODIMENTS

Preferred embodiments of a medical image processing apparatus, a medical image processing method, and a program according to the present invention will be described below with reference to the attached drawings.

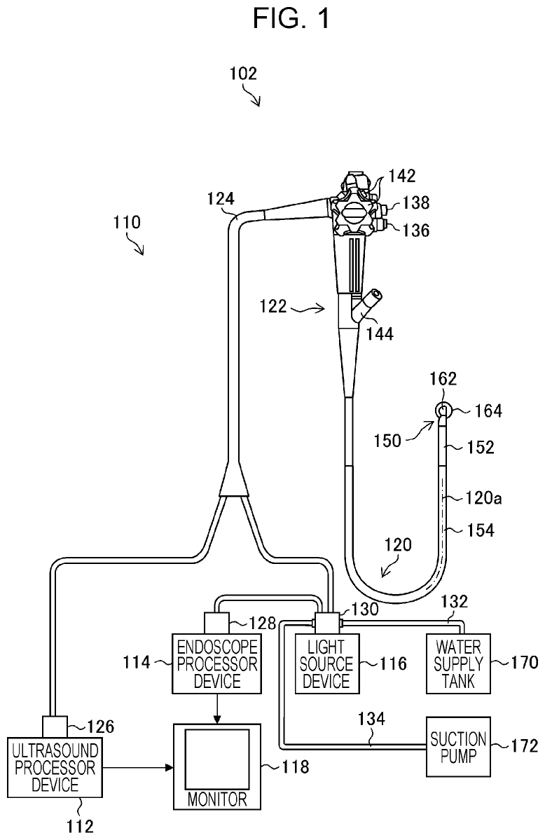

FIG. 1 is a schematic diagram illustrating an overall configuration of an ultrasonic endoscope system equipped with a medical image processing apparatus according to the present invention.

As illustrated in FIG. 1, an ultrasonic endoscope system 102 includes an ultrasonic endoscope 110, an ultrasound processor device 112 that generates an ultrasound image, an endoscope processor device 114 that generates an endoscopic image, a light source device 116 that supplies illumination light for illuminating the inside of a body cavity to the ultrasonic endoscope 110, and a monitor (display unit) 118 that displays the ultrasound image and the endoscopic image. A description of a case where an ultrasound image, which is an example of a medical image, is processed will be given below.

The ultrasonic endoscope 110 includes an insertion part 120 that is inserted into the body cavity of a subject, a hand operation part 122 that is connected to the proximal end portion of the insertion part 120 and is operated by an operator, and a universal cord 124 that has one end connected to the hand operation part 122. At the other end of the universal cord 124, an ultrasonic connector 126 connected to the ultrasound processor device 112, an endoscope connector 128 connected to the endoscope processor device 114, and a light source connector 130 connected to the light source device 116 are provided.

The ultrasonic endoscope 110 is connected to the ultrasound processor device 112, the endoscope processor device 114, and the light source device 116 with the connectors 126, 128, and 130 therebetween so as to be detachable. To the light source connector 130, an air/water supply tube 132 and a suction tube 134 are connected.

The monitor 118 receives video signals generated by the ultrasound processor device 112 and the endoscope processor device 114 and displays an ultrasound image and an endoscopic image. The ultrasound image and the endoscopic image can be displayed such that, for example, only one of the images is displayed on the monitor 118 by switching between the images as appropriate or both of the images are simultaneously displayed.

On the hand operation part 122, an air/water supply button 136 and a suction button 138 are arranged in parallel and a pair of angle knobs 142 and a treatment tool insertion port 144 are provided.

The insertion part 120 has a distal end, a proximal end, and a longitudinal axis 120a and is constituted by a distal end main body 150 formed of a hard material, a bending part 152 connected to the proximal end side of the distal end main body 150, and a soft part 154 that connects the proximal end side of the bending part 152 and the distal end side of the hand operation part 122, that is long and narrow, and that has flexibility, in this order from the distal end side. That is, the distal end main body 150 is provided on the distal end side of the insertion part 120 in the direction of the longitudinal axis 120a. The bending part 152 is remotely operated and bent in response to rotation of the pair of angle knobs 142 provided on the hand operation part 122. Accordingly, the distal end main body 150 can be oriented in a desired direction.

To the distal end main body 150, an ultrasound probe 162 and a pouch-like balloon 164 in which the ultrasound probe 162 is wrapped are attached. The balloon 164 can be inflated or deflated when water is supplied from a water supply tank 170 or water in the balloon 164 is sucked by a suction pump 172. The balloon 164 can be inflated until it comes into contact with the interior wall of a body cavity in order to prevent attenuation of ultrasound and an ultrasonic echo (echo signal) during an ultrasonic observation.

To the distal end main body 150, an endoscopic observation unit that is not illustrated and that has an observation unit including an object lens, an imaging element, and so on and an illumination unit is attached. The endoscopic observation unit is provided behind the ultrasound probe 162 (on a side closer to the hand operation part 122).

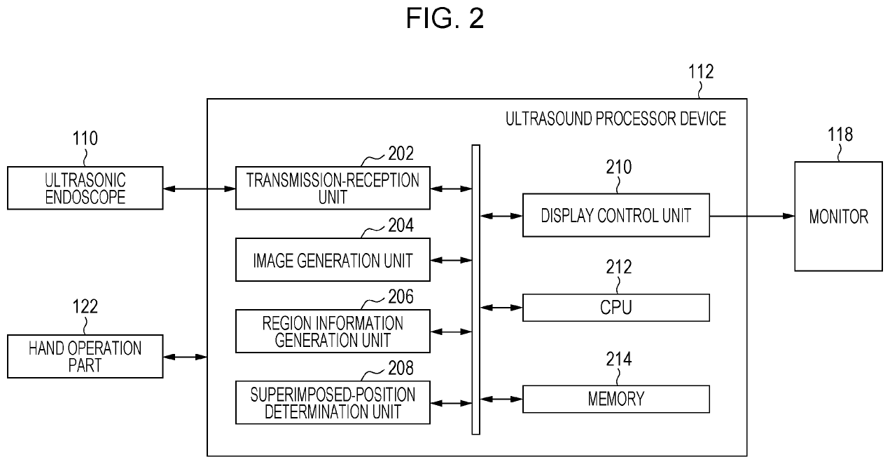

FIG. 2 is a block diagram illustrating an embodiment of the ultrasound processor device 112.

The ultrasound processor device 112 illustrated in FIG. 2 recognizes on the basis of sequentially acquired time-series ultrasound images, the position and category classification of a region of interest in the ultrasound images and notifies a user (a doctor or the like) of information indicating the recognition results. The ultrasound processor device 112 functions as an image processing apparatus that performs image processing on an ultrasound image.

The ultrasound processor device 112 illustrated in FIG. 2 is constituted by a transmission-reception unit 202, an image generation unit 204, a region information generation unit 206, a superimposed-position determination unit 208, a display control unit 210, a CPU (central processing unit) 212, and a memory 214, and processing by each unit is performed by one or more processors not illustrated.

The CPU 212 operates on the basis of various programs including an ultrasound image processing program stored in the memory 214, centrally controls the transmission-reception unit 202, the image generation unit 204, the region information generation unit 206, the superimposed-position determination unit 208, the display control unit 210, and the memory 214, and functions as part of these units.

An ultrasound image acquisition unit (image acquisition unit) performs an image acquisition process. The transmission-reception unit 202 and the image generation unit 204, which functions as the ultrasound image acquisition unit, sequentially acquire time-series ultrasound images.

The transmission-reception unit 202 includes a transmission unit that generates a plurality of driving signals to be applied to a plurality of ultrasonic transducers of the ultrasound probe 162 of the ultrasonic endoscope 110, gives the plurality of driving signals, respective delay times on the basis of a transmission delay pattern selected by a scan control unit not illustrated, and applies the plurality of driving signals to the plurality of ultrasonic transducers.

The transmission-reception unit 202 includes a reception unit that amplifies a plurality of detection signals respectively output from the plurality of ultrasonic transducers of the ultrasound probe 162 and converts the detection signals that are analog signals to digital detection signals (which are also referred to as RF (radio frequency) data). The RF data is input to the image generation unit 204.

The image generation unit 204 gives the plurality of detection signals indicated by the RF data, respective delay times on the basis of a reception delay pattern selected by the scan control unit and adds up the detection signals to thereby perform a reception focus process. By this reception focus process, sound-ray data in which the focus of an ultrasonic echo is narrowed down is formed.

The image generation unit 204 further corrects the sound-ray data for attenuation based on a distance in accordance with the depth of the position of reflection of ultrasound, with STC (sensitivity time-gain control), subsequently generates envelope data by performing an envelope detection process with, for example, a low-pass filter, and stores envelope data for one frame, or more preferably a plurality of frames, in a cine memory not illustrated. The image generation unit 204 performs preprocessing including log (logarithmic) compression and a gain adjustment for the envelope data stored in the cine memory and generates a B-mode image.

As described above, the transmission-reception unit 202 and the image generation unit 204, which function as the ultrasound image acquisition unit, sequentially acquire time-series B-mode images (hereinafter referred to as “ultrasound images”).

The region information generation unit 206 performs a region information generation process and performs a process of detecting regions of interest in an ultrasound image on the basis of the ultrasound image and a process of classifying the regions of interest into a plurality of categories (types) by estimation on the basis of the ultrasound image. The region information generation unit 206 performs these processes to thereby generate region information that includes the positions and category classifications of the regions of interest. The region information generation unit 206 can generate the region information by various methods. For example, the region information generation unit 206 may generate the region information by using AI (artificial intelligence). At least one of the regions of interest is an anatomical region. Further, at least one of the regions of interest may include an annotation drawn by the user on the ultrasound image. Although a case where region information is generated by the region information generation unit 206 is described in this example, the ultrasound processor device 112 may acquire externally generated region information (region information acquisition process).

In category classification performed by the region information generation unit 206, for example, classification according to the type of an organ detected as a region of interest in the ultrasound image (a tomographic image of a B-mode image) is performed. For example, the region information generation unit 206 classifies a detected region of interest as the pancreas (indicated as “Panc” in the figures), the main pancreatic duct (indicated as “MPD” in the figures), the superior mesenteric vein (indicated as “SMV” in the figures), or the gallbladder (indicated as “GB” in the figures). The category classification is superimposed on the ultrasound image and displayed as graphic information. The graphic information is constituted by text information that indicates the category classification. Specific examples of the graphic information include “Panc”, “MPD”, “SMV”, and “GB”.

The superimposed-position determination unit 208 performs a superimposed-position determination process to determine the superimposed positions of pieces of graphic information. The superimposed-position determination unit 208 determines, on the basis of the relative positional relationship between a plurality of regions of interest, the superimposed position (display position) of each of the pieces of graphic information to be displayed by the display control unit 210. Specifically, the superimposed-position determination unit 208 determines the superimposed position of a piece of graphic information of at least one region of interest among the plurality of regions of interest on the basis of the positions of the other regions of interest among the plurality of regions of interest. That is, the superimposed-position determination unit 208 chooses as the superimposed position of a piece of graphic information of one region of interest, a position other than the positions of the other regions of interest such that the piece of graphic information of the one region of interest does not overlap the other regions of interest. Accordingly, the superimposed-position determination unit 208 can determine the superimposed position of the piece of graphic information such that the piece of graphic information does not overlap the regions of interest. Further, the superimposed-position determination unit 208 determines the superimposed position of a piece of graphic information of at least one region of interest among the plurality of regions of interest on the basis of the superimposed positions of pieces of graphic information of the other regions of interest among the plurality of regions of interest. That is, the superimposed-position determination unit 208 chooses as the superimposed position of a piece of graphic information of one region of interest, a position other than the positions of pieces of graphic information of the other regions of interest such that the piece of graphic information of the one region of interest does not overlap the pieces of graphic information of the other regions of interest. Accordingly, the superimposed-position determination unit 208 can superimpose the pieces of graphic information on the ultrasound image such that the pieces of graphic information do not overlap each other.

The display control unit 210 performs a display control process and causes the monitor 118, which is the display unit, to display an ultrasound image. Further, the display control unit 210 superimposes graphic information on the ultrasound image and causes the monitor 118 to display the graphic information. The display control unit 210 displays the graphic information on the monitor 118 on the basis of the position determined by the superimposed-position determination unit 208.

A display form of the ultrasound image displayed on the monitor 118 by the display control unit 210 and the graphic information superimposed on the ultrasound image and displayed will now be specifically described.

First, display of a region of interest detected by the region information generation unit and a bounding box that indicates the bounds of the region of interest will be described.

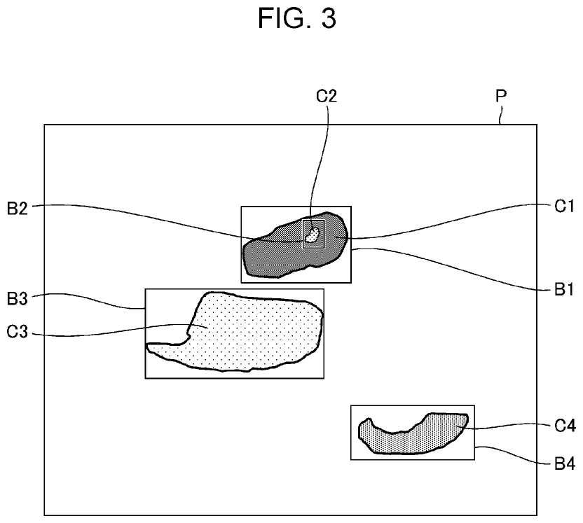

FIG. 3 is a diagram illustrating an example of display of regions of interest detected by the region information generation unit 206 and bounding boxes corresponding to the detected regions of interest.

The display control unit 210 causes the monitor 118 to display an ultrasound image P. The region information generation unit 206 detects a region of interest C1, a region of interest C2, a region of interest C3, and a region of interest C4 in the ultrasound image P and generates information (region information) regarding the positions of the region of interest C1, the region of interest C2, the region of interest C3, and the region of interest C4. Based on the region information, the display control unit 210 superimposes information (bounding boxes) that indicates the bounds of the region of interest C1, the region of interest C2, the region of interest C3, and the region of interest C4 on the ultrasound image P and causes the monitor 118 to display the information. Specifically, the display control unit 210 causes the monitor 118 to display a bounding box B1 corresponding to the region of interest C1, a bounding box B2 corresponding to the region of interest C2, a bounding box B3 corresponding to the region of interest C3, and a bounding box B4 corresponding to the region of interest C4. The bounds of the regions of interest C1 to C4 are thus highlighted by enclosing the regions of interest C1 to C4 in the bounding boxes B1 to B4 to thereby allow the user to easily recognize the positions of the regions of interest C1 to C4.

Display of pieces of graphic information of the regions of interest C1 to C4 will now be described.

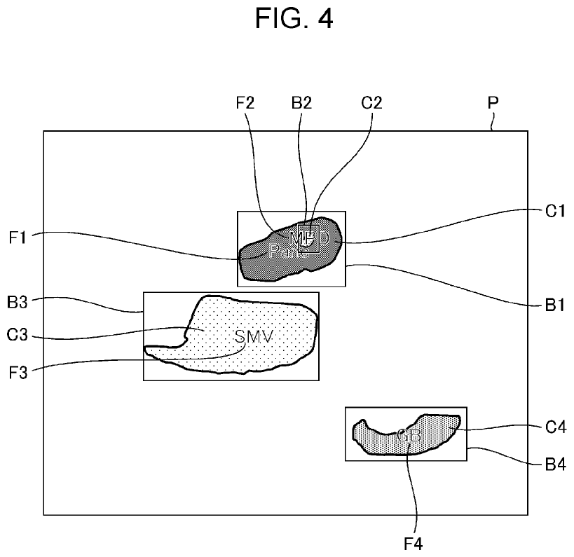

FIG. 4 is a diagram for explaining an example of display of graphic information in the related art.

In the related art, each of the pieces of graphic information of the regions of interest C1 to C4 is often displayed at a predetermined position. For example, graphic information is displayed at the center of a bounding box that indicates the position of the region of interest. Specifically, graphic information (“Panc”) F1 of the region of interest C1 is displayed at the center position of the bounding box B1, graphic information (“MPD”) F2 of the region of interest C2 is displayed at the center position of the bounding box B2, graphic information (“SMV”) F3 of the region of interest C3 is displayed at the center position of the bounding box B3, and graphic information (“GB”) F4 of the region of interest C4 is displayed at the center position of the bounding box B4. As described above, in the related art, graphic information is often displayed at a predetermined position (for example, the center position of the bounding box) regardless of the positions of regions of interest. However, when each of the pieces of graphic information is displayed at the center position of a corresponding bounding box, the pieces of graphic information overlap each other depending on the positions of the detected regions of interest and are difficult to be viewed. For example, in the case illustrated in FIG. 4, the graphic information F1 and the graphic information F2 are displayed close to each other, and the graphic information F1 is displayed while overlapping the bounding box B2 and is difficult to be viewed. Further, when graphic information is thus displayed at the center position of the bounding box, a small region of interest may be difficult to be viewed because of the graphic information. In the case illustrated in FIG. 4, the graphic information F2 is displayed while overlapping the region of interest C2, and the region of interest C2 is difficult to be viewed.

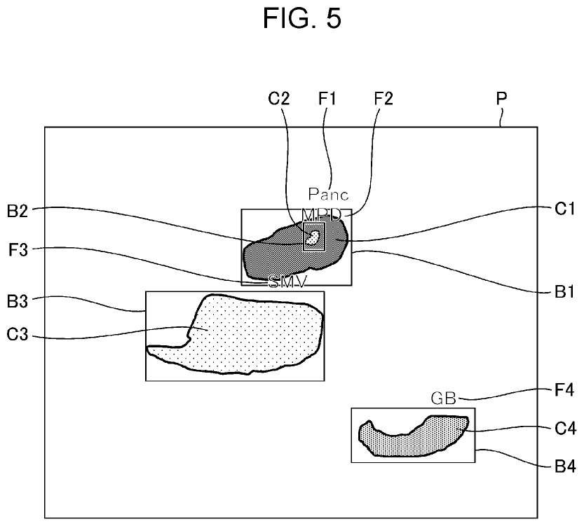

FIG. 5 is a diagram for explaining another example of display of graphic information in the related art.

FIG. 5 illustrates an example in which graphic information is displayed at a predetermined position outside the bounding box. Even when graphic information is displayed outside the bounding box, the graphic information is displayed at a position so as to allow the user to clearly grasp the relationship with the corresponding region of interest. For example, graphic information is displayed near the corresponding bounding box along the bounding box. In the case illustrated in FIG. 5, graphic information is displayed at an upper right position above the bounding box in the figure along the bounding box. Graphic information is thus displayed outside the bounding box to thereby avoid a situation where the graphic information F2 is displayed while overlapping the small region of interest C2 and the region of interest C2, which is an observation target, is hidden behind the graphic information F2 as described with reference to FIG. 4. However, the pieces of graphic information F1 to F4 are displayed at the predetermined positions (upper right positions) above the bounding boxes B1 to B4, and therefore, pieces of graphic information may overlap each other or a piece of graphic information may be superimposed on a region of interest and displayed depending on the positions of the detected regions of interest. For example, the graphic information F2 and the graphic information F3 are each displayed while overlapping the region of interest C1 and the bounding box B1.

As described above, when each of the pieces of graphic information is displayed at the predetermined position, pieces of graphic information may overlap each other or a piece of graphic information may be displayed while overlapping a region of interest depending on the positions of the detected regions of interest.

According to the present invention, a situation where pieces of graphic information overlap each other or a piece of graphic information is displayed while overlapping a region of interest as described above is avoided to allow easily viewable display of pieces of graphic information and regions of interest.

### First Embodiment

A first embodiment of the present invention will now be described.

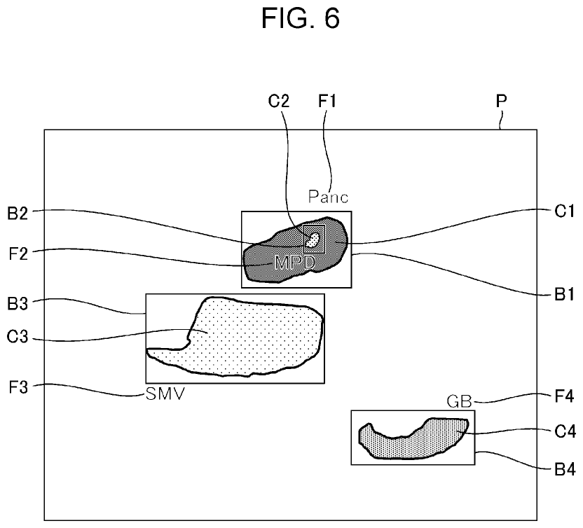

FIG. 6 is a diagram illustrating an example of display of graphic information of this embodiment.

In this embodiment, the superimposed-position determination unit 208 determines the superimposed position of graphic information in accordance with the positions of regions of interest. This can avoid a situation where pieces of graphic information overlap each other and a situation where a piece of graphic information overlaps a region of interest.

Even when each of the graphic information F1 and the graphic information F4 is displayed at the predetermined position, the graphic information F1 and the graphic information F4 do not overlap another region of interest or another piece of graphic information, and therefore, the superimposed-position determination unit 208 chooses the predetermined position (the upper right position in the figure outside each bounding box) as the superimposed positions of the graphic information F1 and the graphic information F4. Meanwhile, when displayed at the predetermined upper right position above the bounding box, the graphic information F3 overlaps the region of interest C1 and the bounding box B1 and hinders observation of the region of interest C1, and therefore, the superimposed-position determination unit 208 determines the position so as to display the graphic information F3 at a lower left position below the bounding box. When displayed at the predetermined upper right position above the bounding box, the graphic information F2 overlaps the graphic information F1, and therefore, the superimposed-position determination unit 208 determines the position so as to display the graphic information F2 at a lower left position below the bounding box.

As described above, the superimposed-position determination unit 208 determines the superimposed position of the graphic information F3 in accordance with the positions of the regions of interest C1, C2, and C4 and the positions of the pieces of graphic information F1, F2, and F4. Further, the superimposed-position determination unit 208 determines the superimposed position of the graphic information F2 in accordance with the positions of the regions of interest C1, C3, and C4 and the positions of the pieces of graphic information F1, F3, and F4. Accordingly, the pieces of graphic information F1 to F4 are displayed on the monitor 118 so as to be easily viewable without the pieces of graphic information overlapping each other or overlapping regions of interest.

A medical image processing method using the medical image processing apparatus will now be described. In the medical image processing method, each of the steps is performed by a processor executing a program.

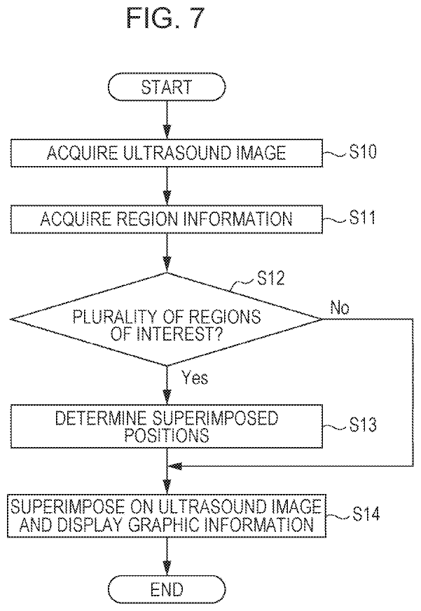

FIG. 7 is a flowchart illustrating the medical image processing method.

First, the transmission-reception unit 202 and the image generation unit 204, which function as the ultrasound image acquisition unit, acquire an ultrasound image (step S10: image acquisition step). Subsequently, the region information generation unit 206 generates region information that includes the positions and category classifications of one or more regions of interest included in the ultrasound image, and the region information is acquired (step S11: region information acquisition step). Next, the superimposed-position determination unit 208 determines whether a plurality of detected regions of interest are present in the region information (step S12). If a single region of interest is present in the region information, the superimposed-position determination unit 208 superimposes a piece of graphic information on the ultrasound image at the predetermined position and causes the graphic information to be displayed. On the other hand, if the region information has a plurality of pieces of regions of interest, the superimposed-position determination unit 208 determines the superimposed positions of pieces of graphic information on the basis of the relative positional relationship between the plurality of regions of interest (step S13: superimposed-position determination step). The display control unit 210 superimposes the one or more pieces of graphic information on the ultrasound image on the basis of the determined superimposed positions and causes the monitor 118 to display the pieces of graphic information (step S14: display control step).

As described above, according to this embodiment, when region information has information regarding a plurality of regions of interest, the superimposed positions of pieces of graphic information to be displayed by the display control unit 210 are determined on the basis of the relative positional relationship between the plurality of regions of interest, and the pieces of graphic information are displayed. Accordingly, in this embodiment, even when a plurality of regions of interest are detected, the regions of interest and pieces of graphic information can be displayed so as to be easily viewable.

### Second Embodiment

A second embodiment of the present invention will now be described. In this embodiment, the inclusion relationship between detected regions of interest is acquired, and pieces of graphic information are displayed on the basis of the inclusion relationship.

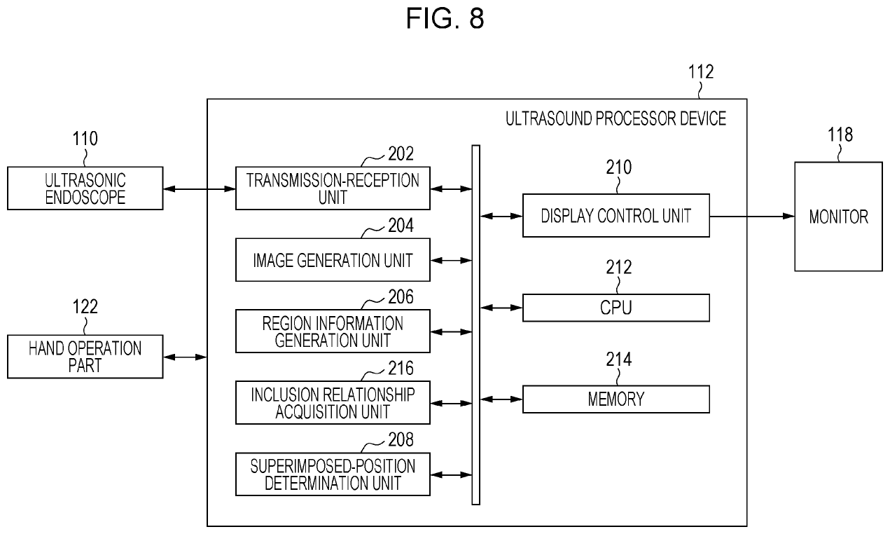

FIG. 8 is a block diagram illustrating an embodiment of the ultrasound processor device 112 of this embodiment. Note that a part for which a description has been given with reference to FIG. 2 is assigned the same reference numeral and a description thereof will be omitted.

The ultrasound processor device 112 illustrated in FIG. 8 is constituted by the transmission-reception unit 202, the image generation unit 204, the region information generation unit 206, an inclusion relationship acquisition unit 216, the superimposed-position determination unit 208, the display control unit 210, the CPU (central processing unit) 212, and the memory 214, and processing by each unit is performed by one or more processors not illustrated.

The inclusion relationship acquisition unit 216 performs an inclusion relationship acquisition process to acquire inclusion relationship information about a plurality of regions of interest on the basis of region information. Specifically, the inclusion relationship acquisition unit 216 acquires the inclusion relationship between regions when, for example, one region is included in another region, on the basis of region information. For example, when the region information generation unit 206 detects the regions of the pancreas and the main pancreatic duct, the inclusion relationship acquisition unit 216 acquires an inclusion relationship that the main pancreatic duct is included in the pancreas, on the basis of the positional relationship between the detected regions of interest or the recognized category classifications of the detected regions of interest. The inclusion relationship acquisition unit 216 can acquire the inclusion relationship between regions of interest by various methods. For example, the inclusion relationship acquisition unit 216 stores in advance table data that indicates inclusion relationships, and acquires an inclusion relationship on the basis of the table data and the category classifications. The superimposed-position determination unit 208 determines the superimposed positions of pieces of graphic information on the basis of the inclusion relationship. The display control unit 210 can change the display form of the pieces of graphic information on the basis of the inclusion relationship.

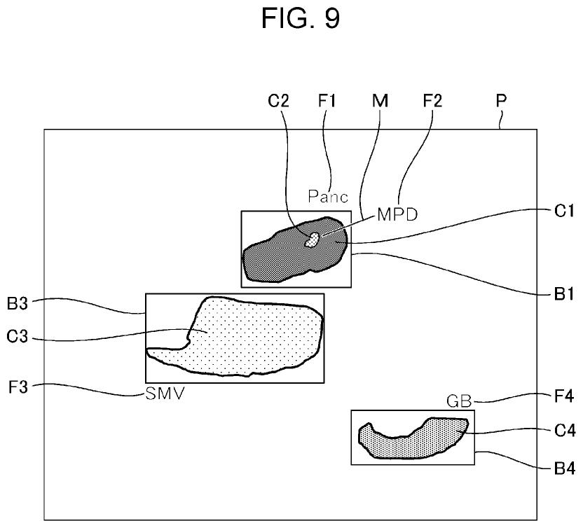

FIG. 9 is a diagram illustrating an example display form to which the present invention is applied. Note that a part for which a description has been given with reference to FIG. 6 is assigned the same reference numeral and a description thereof will be omitted.

The inclusion relationship acquisition unit 216 acquires region information about the ultrasound image P1 and acquires an inclusion relationship that the region of interest C2 is included in the region of interest C1. The region information has category classifications, based on which the region of interest C1 is the pancreas and the region of interest C2 is the main pancreatic duct, and therefore, the inclusion relationship acquisition unit 216 acquires on the basis of the stored table data, inclusion relationship information indicating that the region of interest C2 is included in the region of interest C1.

In the example illustrated in FIG. 9, the display control unit 210 displays the graphic information F2 and the region of interest C2 in association with each other with a leader line M. That is, according to the inclusion relationship information, the region of interest C2 is included in the region of interest C1, and therefore, when the graphic information F2 is displayed at the predetermined position (the upper right position above the bounding box B2), the graphic information F2 is displayed while overlapping the region of interest C1. Therefore, the superimposed-position determination unit 208 chooses as the superimposed position of the graphic information F2, a position outside the bounds of the region of interest C1. The superimposed-position determination unit 208 determines on the basis of the inclusion relationship, whether to display a leader line, and the display control unit 210 displays the leader line M so as to indicate the correspondence between the graphic information F2 and the region of interest C2.

In the example illustrated in FIG. 9, the position of the region of interest C2 is indicated by the leader line M, and therefore, the bounding box B2 is not displayed. This can avoid a situation where the bounding box B2 is displayed while overlapping the region of interest C1 and reduce the complexity of the entire image display.

As described above, the superimposed-position determination unit 208 can also determine the superimposed position of graphic information on the basis of the inclusion relationship. When the superimposed position of graphic information is thus determined on the basis of the inclusion relationship, the graphic information can be prevented from being displayed while overlapping a region of interest. When graphic information is displayed away from the corresponding region of interest, the display control unit 210 causes a leader line to be displayed to thereby allow the correspondence between the graphic information and the region of interest to be indicated.

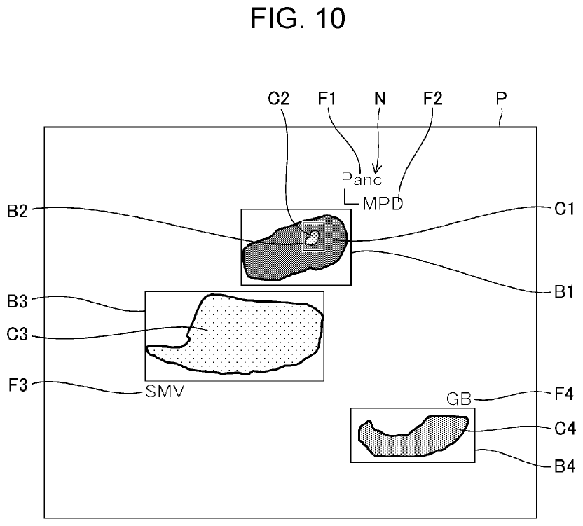

FIG. 10 is a diagram illustrating another example display form of this embodiment. As described with reference to FIG. 9, the superimposed-position determination unit 208 has the inclusion relationship between the region of interest C1 and the region of interest C2.

The display control unit 210 displays the graphic information F1 and the graphic information F2 with nested display N on the basis of the inclusion relationship between the region of interest C1 and the region of interest C2. When the graphic information F1 and the graphic information F2 are thus displayed with the nested display N, the graphic information F2 can be prevented from being displayed while overlapping the region of interest C1 and the inclusion relationship between the graphic information F1 and the graphic information F2 can be indicated.

As described above, the display control unit 210 can also display pieces of graphic information with the nested display on the basis of the inclusion relationship. When pieces of graphic information having an inclusion relationship are thus displayed by using the nested display, a piece of graphic information can be prevented from being displayed while overlapping a region of interest. With the nested display, the inclusion relationship between pieces of graphic information can be presented to the user.

### Third Embodiment

A third embodiment of the present invention will now be described. In this embodiment, an adjustment is made for a shift of the superimposed position of graphic information between image frames that constitute a motion picture.

The transmission-reception unit 202 and the image generation unit 204 perform an ultrasound image acquisition process to thereby sequentially acquire successive time-series ultrasound images.

The region information generation unit 206 generates region information for each ultrasound image of the successive ultrasound images. The superimposed-position determination unit 208 determines on a per ultrasound-image basis, the superimposed position of graphic information on the basis of the region information generated for each ultrasound image by the region information generation unit 206.

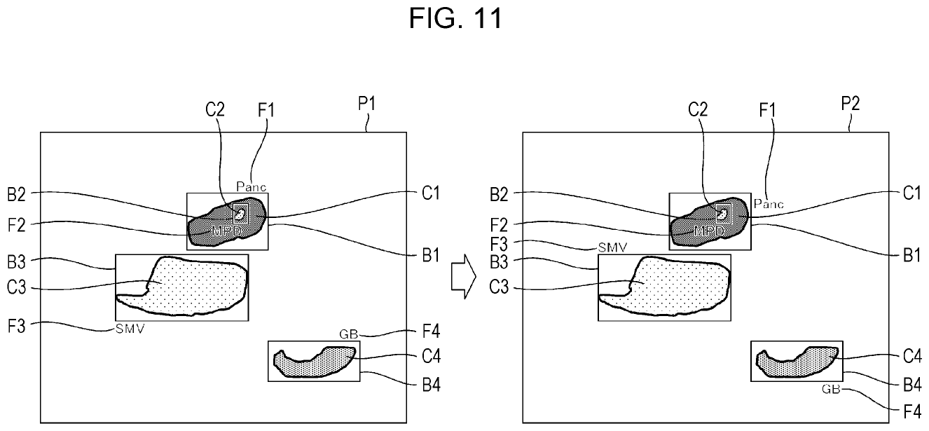

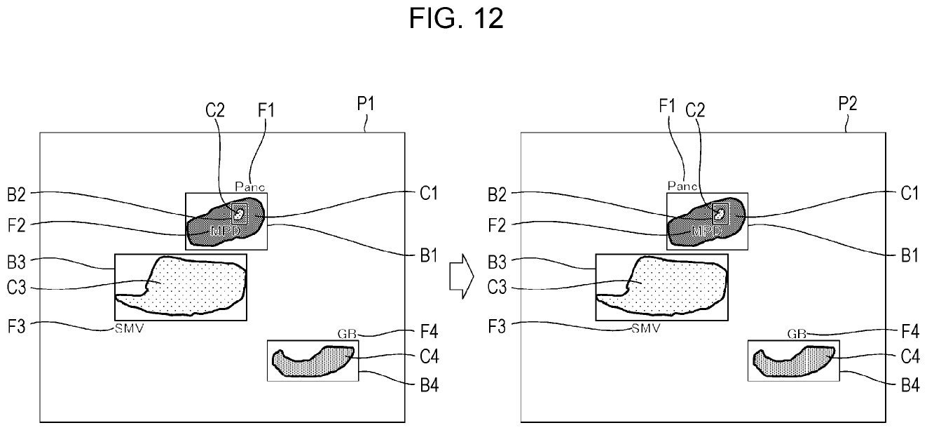

FIG. 11 and FIG. 12 are diagrams for explaining display of graphic information on an ultrasound image P1 and an ultrasound image P2.

The ultrasound image P1 and the ultrasound image P2 are successive time-series images, and for the ultrasound image P1 and the ultrasound image P2, the superimposed-position determination unit 208 determines the superimposed positions of the pieces of graphic information F1 to F4. For each of the ultrasound image P1 and the ultrasound image P2, the superimposed-position determination unit 208 determines the superimposed positions of the pieces of graphic information F1 to F4 in accordance with the positions of the regions of interest C1 to C4 and the positions of the pieces of graphic information F1 to F4.

Between the ultrasound image P1 and the ultrasound image P2, the graphic information F1, the graphic information F3, and the graphic information F4 move. Specifically, the graphic information F1 is located at the upper right position above the bounding box B1 in the ultrasound image P1 but is located to the right of the bounding box B1 in the ultrasound image P2. The graphic information F3 is located at the lower left position below the bounding box B3 in the ultrasound image P1 but is located at the upper left position above the bounding box B3 in the ultrasound image P2. The graphic information F4 is located at the upper right position above the bounding box B4 in the ultrasound image P1 but is located at the lower right position below the bounding box B4 in the ultrasound image P2. When the superimposed positions of the pieces of graphic information are changed to a large degree between the successive time-series ultrasound image P1 and ultrasound image P2, visibility is compromised. Therefore, in this embodiment, the superimposed positions are determined such that the positions of the pieces of graphic information do not change to a large degree between the ultrasound image P1 and the ultrasound image P2. Specifically, the superimposed-position determination unit 208 determines the superimposed positions of the pieces of graphic information in the ultrasound image P2 (current ultrasound image) on the basis of the superimposed positions of the pieces of graphic information in the ultrasound image P1 (past ultrasound image). For example, the superimposed-position determination unit 208 chooses as the superimposed position of graphic information, a position in a region within a distance of a first threshold value from the superimposed position of the graphic information in the ultrasound image P1.

FIG. 12 is a diagram illustrating an example display form on the monitor 118 of this embodiment.

In the case illustrated in FIG. 12, the superimposed-position determination unit 208 chooses as the superimposed position of graphic information, a position in a region within a distance of the first threshold value. Specifically, each of the pieces of graphic information F1, F3, and F4 is superimposed at a position that is shifted from the position in the ultrasound image P1 and that is in a region within a distance of the first threshold value. When the superimposed-position determination unit 208 thus chooses as the superimposed position of graphic information, a position in a region within a distance of the first threshold value from the superimposed position of the graphic information in the ultrasound image P1, the superimposed position of the graphic information does not change to a large degree between the frames and easily viewable display can be provided.

### Fourth Embodiment

A fourth embodiment of the present invention will now be described. In this embodiment, when an annotation is drawn by the user while the ultrasound image P is displayed on the monitor 118, graphic information is displayed away from the annotation region.

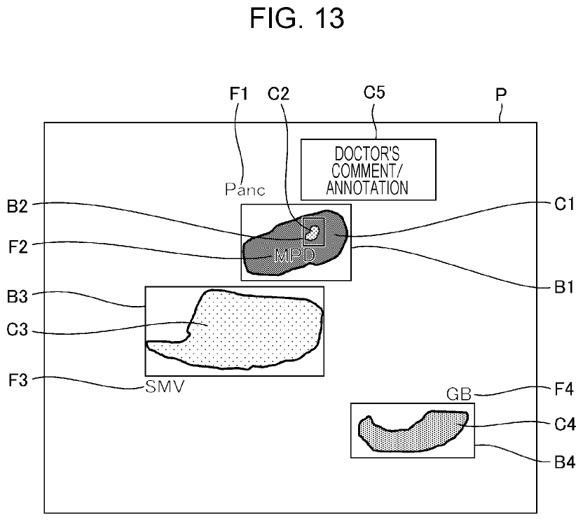

FIG. 13 is a diagram for explaining an example display form of this embodiment. Note that a part for which a description has been given with reference to FIG. 6 is assigned the same reference numeral and a description thereof will be omitted.

While the ultrasound image P is displayed on the monitor 118, the user may directly add an annotation to the ultrasound image P. The user can add an annotation to the ultrasound image by using an operation unit (not illustrated) connected to the ultrasound processor device.

The region information generation unit 206 detects a region to which the annotation is added, as a region of interest C5. In this case, the category classification of the region of interest C5 is “annotation”. The region information generation unit 206 generates region information including the position and category classification (annotation) of the region of interest C5. For the annotation that is detected as the region of interest C5, graphic information is not displayed.

The superimposed-position determination unit 208 superimposes pieces of graphic information on the basis of the region information such that the pieces of graphic information do not overlap the regions of interest C1 to C5. Specifically, when superimposed at the predetermined position (the upper right position above the bounding box B1), the graphic information F1 overlaps the region of interest C5, and therefore, the graphic information F1 is superimposed at the upper left position above the bounding box B1. An annotation that is added while the ultrasound image is displayed is also displayed so as not to overlap graphic information as described above to thereby allow the annotation to be easily viewable.

### Others

Although a description regarding an ultrasound image, which is an example of the medical image, has been given above, the medical image to which the present invention is applied is not limited to ultrasound images. For example, the present invention is applied also to an endoscopic image, which is another example of the medical image.

In the embodiments described above, the hardware configuration of the processing units that perform various types of processing is implemented as various processors as described below. The various processors include a CPU (central processing unit), which is a general-purpose processor executing software (program) to function as various processing units, a programmable logic device (PLD), such as an FPGA (field-programmable gate array), which is a processor for which the circuit configuration can be changed after manufacturing, and a dedicated electric circuit, such as an ASIC (application-specific integrated circuit), which is a processor having a circuit configuration that is designed only for performing a specific process.

One processing unit may be configured as one of the various processors or two or more processors of the same type or different types (for example, a plurality of FPGAs or a combination of a CPU and an FPGA). Further, a plurality of processing units may be configured as one processor. As the first example of configuring a plurality of processing units as one processor, a form is possible in which one or more CPUs and software are combined to configure one processor, and the processor functions as the plurality of processing units, a representative example of which is a computer, such as a client or a server. As the second example thereof, a form is possible in which a processor is used in which the functions of the entire system including the plurality of processing units are implemented as one IC (integrated circuit) chip, a representative example of which is a system on chip (SoC). As described above, regarding the hardware configuration, the various processing units are configured by using one or more of the various processors described above.

Further, the hardware configuration of the various processors is more specifically an electric circuit (circuitry) in which circuit elements, such as semiconductor elements, are combined.

The configurations and functions described above can be implemented as any hardware, software, or a combination thereof as appropriate. For example, the present invention is applicable to a program for causing a computer to perform the above-described processing steps (processing procedure), a computer-readable recording medium (non-transitory recording medium) to which the program is recorded, or a computer in which the program can be installed.

Although an example of the present invention has been described above, the present invention is not limited to the embodiments described above, and various modifications can be made without departing from the gist of the present invention as a matter of course.

### REFERENCE SIGNS LIST

102 ultrasonic endoscope system
110 ultrasonic endoscope
112 ultrasound processor device
114 endoscope processor device
116 light source device
118 monitor
120 insertion part
120a longitudinal axis
122 hand operation part
124 universal cord
126 ultrasonic connector
128 endoscope connector
130 light source connector
132 tube
134 tube
136 air/water supply button
138 suction button
142 angle knob
144 treatment tool insertion port
150 distal end main body
152 bending part
154 soft part
162 ultrasound probe
164 balloon
170 water supply tank
172 suction pump
202 transmission-reception unit
204 image generation unit
206 region information generation unit
208 superimposed-position determination unit
210 display control unit
212 CPU
214 memory
216 inclusion relationship acquisition unit

What is claimed is:

1. A medical image processing apparatus comprising: a processor,
the processor being configured to perform:
an image acquisition process of acquiring a medical image;
a region information acquisition process of acquiring region information regarding a plurality of regions of interest included in the medical image, the region information including positions and category classifications of the plurality of regions of interest;
a display control process of superimposing a plurality of pieces of graphic information that indicate the category classifications of the plurality of regions of interest on the medical image and causing a display unit to display the plurality of pieces of graphic information; and
a superimposed-position determination process of determining superimposed positions of the plurality of pieces of graphic information to be displayed by the display control process, on the basis of a relative positional relationship between the plurality of regions of interest.

2. The medical image processing apparatus according to claim 1, wherein the processor is configured to perform a region information generation process of generating the region information by detecting the plurality of regions of interest included in the medical image and estimating the category classifications of the plurality of detected regions of interest.

3. The medical image processing apparatus according to claim 1, wherein at least one of the plurality of regions of interest is an anatomical region.

4. The medical image processing apparatus according to claim 1, wherein at least one of the plurality of regions of interest is an annotation drawn by a user on the medical image.

5. The medical image processing apparatus according to claim 1, wherein in the superimposed-position determination process, a superimposed position of a piece of graphic information of at least one region of interest among the plurality of regions of interest is determined on the basis of a position of another region of interest among the plurality of regions of interest.

6. The medical image processing apparatus according to claim 1, wherein in the superimposed-position determination process, a superimposed position of a piece of graphic information of at least one region of interest among the plurality of regions of interest is determined on the basis of a superimposed position of a piece of graphic information of another region of interest among the plurality of regions of interest.

7. The medical image processing apparatus according to claim 1, wherein in the image acquisition process, a plurality of successive time-series medical images are acquired.

8. The medical image processing apparatus according to claim 7, wherein in the superimposed-position determination process, among the plurality of medical images, a superimposed position of graphic information in a current medical image is determined on the basis of a superimposed position of the graphic information in a past medical image.

9. The medical image processing apparatus according to claim 8, wherein in the superimposed-position determination process, as the superimposed position of the graphic information in the current medical image, a position in a region that is within a distance of a first threshold value from the superimposed position of the graphic information in the past medical image is chosen.

10. The medical image processing apparatus according to claim 1, wherein in the display control process, a leader line that extends up to a superimposed position of a piece of graphic information of at least one region of interest among the plurality of regions of interest is displayed.

11. The medical image processing apparatus according to claim 1, wherein the processor is configured to perform an inclusion relationship acquisition process of acquiring inclusion relationship information about the plurality of regions of interest on the basis of the region information, and
in the superimposed-position determination process, the superimposed positions of the plurality of pieces of graphic information are determined on the basis of the inclusion relationship information.

12. The medical image processing apparatus according to claim 11, wherein in the superimposed-position determination process, whether to display a leader line for at least one region of interest among the plurality of regions of interest is determined on the basis of the inclusion relationship information to switch between display and non-display.

13. The medical image processing apparatus according to claim 12, wherein in the display control process, pieces of graphic information of regions of interest having an inclusion relationship are displayed in a nested form that indicates the inclusion relationship, on the basis of the inclusion relationship information.

14. The medical image processing apparatus according to claim 1, wherein in the display control process, pieces of information that indicate bounds of the regions of interest are displayed on the basis of the region information.

15. The medical image processing apparatus according to claim 14, wherein the pieces of information indicating the bounds of the regions of interest are bounding boxes, and in the display control process, the pieces of graphic information are displayed so as to correspond to the bounding boxes.

16. The medical image processing apparatus according to claim 1, wherein each of the pieces of graphic information is constituted by text information that indicates a corresponding one of the category classifications.

17. A medical image processing method using a medical image processing apparatus comprising a processor,
the medical image processing method comprising:
an image acquisition step of acquiring a medical image;
a region information acquisition step of acquiring region information regarding a plurality of regions of interest included in the medical image, the region information including positions and category classifications of the plurality of regions of interest;
a display control step of superimposing a plurality of pieces of graphic information that indicate the category classifications of the plurality of regions of interest on the medical image and causing a display unit to display the plurality of pieces of graphic information; and
a superimposed-position determination step of determining superimposed positions of the plurality of pieces of graphic information to be displayed in the display control step, on the basis of a relative positional relationship between the plurality of regions of interest, the steps being performed by the processor.

18. A non-transitory, computer-readable tangible recording medium to which a program for causing, when read by a computer, the computer to execute the medical image processing method according to claim 17 is recorded.
